(19) United States
(12) Patent Application Publication
(10) Pub. No.: US 2025/0285707 A1
(43) Pub. Date: Sep. 11, 2025

(54) METHODS OF GENOTYPING RARE GENETIC VARIANTS

(71) Applicant: Affymetrix, Inc., Carlsbad, CA (US)

(72) Inventors: Teresa Webster, Olga, WA (US); Jeanette Pruzan Schmidt, Palo Alto, CA (US); Orna Mizrahi Man, Eshkhar (IL); Marcos Woehrmann, Fremont, CA (US); Anuradha Mittal, San Jose, CA (US)

(21) Appl. No.: 18/859,967

(22) PCT Filed: Apr. 26, 2023
(86) PCT No.: PCT/US2023/020084

§ 371 Date: Oct. 24, 2024

Related U.S. Application Data
(60) Provisional application No. 63/335,159, filed on Apr. 26, 2022.
(60) Provisional application No. 63/344,020, filed on May 19, 2022.

(51) Int. Cl. G16B 20/20; G06N 20/10; G16B 40/20

(52) U.S. Cl. CPC G16B 20/20; G06N 20/10; G16B 40/20

(57) ABSTRACT

A method of genotyping one or more genetic rare variants in a plurality of nucleic acid samples is described. The method comprises running one or more assays of the plurality of nucleic acid samples using at least one microarray comprising a plurality of probesets and generating assay data. Heterozygous genotypes in the plurality of nucleic acid samples are called and evaluated to identify any rare heterozygous genotype calls that are from a probeset having at least one probe, wherein the number of heterozygous genotype calls for the probeset does not exceed a maximum threshold value. Each identified rare heterozygous genotype call is evaluated to determine whether the identified rare heterozygous genotype call comprises a true rare heterozygous genotype call using a support vector machine prediction model or supervised machine learning classification model comprising a plurality of predictor values for identifying a true rare heterozygous genotype call.

### BACKGROUND

The translation of biomarkers from discovery to routine clinical applications is vital to the future of precision medicine. Yet, applying these discoveries and associating population-specific variants to individual traits, diseases, and potential downstream treatment options remains one of the most challenging barriers to personalized genomics. While large population cohort studies and private sequencing initiatives have revealed thousands of single-nucleotide polymorphisms (SNPs) and structural variants that are implicated in nearly 2,000 human diseases and traits, many of these variants are not within genes and have no known biological function. Better understanding of the role of these variants requires studies that are optimized for population and imputation. These studies must integrate knowledge of diseases, traits, and biological functions using meticulously designed genotyping panels and a reliable, fast, and flexible genotyping platform.

Furthermore, the widespread use of next-generation sequencing (NGS) in clinical settings has led to the identification of many novel genetic variants, including genetic rare variants. Along with the improvement of high throughput sequencing technologies, the genetics community is also showing marked interest for the rare variants/common diseases hypothesis. The Common Disease-Rare Variant Hypothesis (CDRVH) hypothesizes that if a disease with genetic causes is common in the population (a prevalence greater than 1-5%), then the genetic causes—specific genetic errors (genetic variants or disease alleles)—will not necessarily be found to be common in the population as suggested by the competing Common Disease-Common Variant Hypothesis (CDCVH) but rather will be comprised of a multiplicity of risk alleles, each of which is individually rare in the population. It makes this prediction for diseases whose genetic contribution is believed to come from multiple genes simultaneously, polygenic disorders. Autism is thought to be such a disorder.

Genetic databases are now available for interpreting or associating genetic variants for various health conditions. For example, ClinVar (https://www.ncbi.nlm.nih.gov/clinvar/) at the National Center for Biotechnology Information (NCBI) is a freely available archive for interpretations of clinical significance of genetic variants for reported conditions. The database includes germline and somatic variants of any size, type or genomic location. Interpretations are submitted by clinical testing laboratories, research laboratories, locus-specific databases, OMIM®, GeneReviews™, UniProt, expert panels and practice guidelines. See Landrum et al., ClinVar: Public archive of interpretations of clinically relevant variants, Nucleic Acid Re 2016 Jan. 4; Vol. 44 (Database issue): D862-D868 (https://www.ncbi.nlm.nih.gov/pmc/articles/PMC4702865/)

With databases such as ClinVar, genetic rare variants are associated with traits and pathology, and some researchers and health care providers would like to design and use microarrays that detect the occurrence of such rare variants. However, genotyping rare variants is more challenging than genotyping common variants, and false heterozygous (Het) genotypes for rare variants called on microarray platforms can cause problems in the analysis of data by researchers and health care providers.

Very rare variants can be more difficult to genotype on microarray platforms because they use clustering algorithms to identify the genotype. While for common variants, the location and shape of the heterozygous (Het) cluster may provide powerful evidence for the accuracy of Het calls, rare variants often have no other samples in the Het cluster, making the call less robust.

### SUMMARY

A method of genotyping one or more genetic rare variants in a plurality of nucleic acid samples is described. The method comprises running one or more assays of the plurality of nucleic acid samples using at least one microarray comprising a plurality of probesets and generating assay data. Heterozygous genotypes in the plurality of nucleic acid samples are called and evaluated to identify any rare heterozygous genotype calls wherein the number of heterozygous genotype calls for the probeset does not exceed a maximum threshold value. Each identified rare heterozygous genotype call is evaluated to determine whether the identified rare heterozygous genotype call comprises a true rare heterozygous genotype call using a support vector machine prediction model comprising a plurality of predictor values for identifying a true rare heterozygous genotype call.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flowchart illustrating a method for genotyping rare genetic variants according to various embodiments of the present disclosure.

FIG. 2 illustrates genotyping clustering diagrams according to various embodiments of the present disclosure.

FIG. 3 is a flow diagram illustrating a method for rare heterozygous adjustment (RHA) according to various embodiments of the present disclosure.

FIG. 4 is a flow diagram further illustrating a method for rare heterozygous adjustment (RHA) according to various embodiments of the present disclosure.

FIG. 5 illustrates graphs showing acceptable ranges of Z-scores relative to distribution of intensities according to various embodiments of the present disclosure.

FIG. 6 illustrates a graph and a data table showing changes in genotype calling accuracy for representative data sets according to various embodiments of the present disclosure.

FIG. 7 illustrates a graph showing results of an RHA algorithm used in one embodiment of the present disclosure.

FIG. 8 is a flow chart showing a method of genotyping rare variants including a support vector machine analysis according to some embodiments of the present disclosure.

FIG. 9 is a results table for a support vector machine analysis according to some embodiments of the present disclosure.

FIG. 10A illustrates a graph and a data table for various predictors for a support vector machine analysis according to some embodiments of the present disclosure.

FIG. 10B illustrates a graph and a data table for various training set sizes for a support vector machine analysis according to some embodiments of the present disclosure.

FIG. 11 is a test results table for a support vector machine prediction model according to some embodiments of the present disclosure.

FIG. 12 illustrates graphs of test results data for a support vector machine prediction model according to some embodiments of the present disclosure.

FIG. 13 is a flow chart showing a method of genotyping rare variants including a support vector machine analysis according to some embodiments of the present disclosure.

FIG. 14 illustrates a test results table and a graph of test results data for a support vector machine prediction model according to some embodiments of the present disclosure wherein heterozygous calls for an allele were produced by probesets with only one probe for each allele on a microarray.

FIG. 15 is a high-level block diagram of an exemplary apparatus that may be used to implement systems, apparatus and methods described herein.

While the invention is described with reference to the above drawings, the drawings are intended to be illustrative, and other embodiments are consistent with the spirit, and within the scope, of the invention.

### DETAILED DESCRIPTION

To provide a more thorough understanding of the present invention, the following description sets forth numerous specific details, such as specific configurations, parameters, examples, and the like. It should be recognized, however, that such description is not intended as a limitation on the scope of the present invention but is intended to provide a better description of the exemplary embodiments.

Throughout the specification and claims, the following terms take the meanings explicitly associated herein, unless the context clearly dictates otherwise:

The phrase “in one embodiment” as used herein does not necessarily refer to the same embodiment, though it may. Thus, as described below, various embodiments of the invention may be readily combined, without departing from the scope or spirit of the invention.

As used herein, the term “or” is an inclusive “or” operator and is equivalent to the term “and/or,” unless the context clearly dictates otherwise.

The term “based on” is not exclusive and allows for being based on additional factors not described unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of a networked environment where two or more components or devices are able to exchange data, the terms “coupled to” and “coupled with” are also used to mean “communicatively coupled with”, possibly via one or more intermediary devices.

In addition, throughout the specification, the meaning of “a”, “an”, and “the” includes plural references, and the meaning of “in” includes “in” and “on”.

Although some of the various embodiments presented herein constitute a single combination of inventive elements, it should be appreciated that the inventive subject matter is considered to include all possible combinations of the disclosed elements. As such, if one embodiment comprises elements A, B, and C, and another embodiment comprises elements B and D, then the inventive subject matter is also considered to include other remaining combinations of A, B, C, or D, even if not explicitly discussed herein. Further, the transitional term “comprising” means to have as parts or members, or to be those parts or members. As used herein, the transitional term “comprising” is inclusive or open-ended and does not exclude additional, unrecited elements or method steps.

Throughout the following disclosure, numerous references may be made regarding servers, services, interfaces, engines, modules, clients, peers, portals, platforms, or other systems formed from computing devices. It should be appreciated that the use of such terms is deemed to represent one or more computing devices having at least one processor (e.g., ASIC, FPGA, DSP, x86, ARM, ColdFire, GPU, multi-core processors, etc.) configured to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable medium storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, a circuit-switched network, the Internet, LAN, WAN, VPN, or other type of network.

As used in the description herein and throughout the claims that follow, when a system, engine, server, device, module, or other computing element is described as being configured to perform or execute functions on data in a memory, the meaning of “configured to” or “programmed to” is defined as one or more processors or cores of the computing element being programmed by a set of software instructions stored in the memory of the computing element to execute the set of functions on target data or data objects stored in the memory.

It should be noted that any language directed to a computer should be read to include any suitable combination of computing devices or network platforms, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

In various embodiments, the systems and methods described herein may be used to detect one or more types of biological components of interest. These biological components of interest may be any suitable biological target including, but are not limited to, DNA sequences (including cell-free DNA), RNA sequences, genes, oligonucleotides, molecules, proteins, biomarkers, cells (e.g., circulating tumor cells), or any other suitable target biomolecule.

In various embodiments, such biological components may be used in conjunction with various polymerase chain reaction (PCR) methods and systems in applications such as multiplex PCR, viral detection and quantification standards, genotyping, sequencing validation, mutation detection, detection of genetically modified organisms, fetal diagnostics, rare allele detection, and copy number variation.

Embodiments of the present disclosure are generally directed to systems and methods for genotyping one or more rare genetic variants in a plurality of biological samples.

While generally applicable to digital quantification such as PCR, it should be recognized that any other suitable quantification method may be used in accordance with various embodiments described herein. Suitable PCR methods include, but are not limited to, digital PCR, allele-specific PCR, asymmetric PCR, ligation-mediated PCR, multiplex PCR, nested PCR, qPCR, genome walking, and bridge PCR, for example.

As used herein, thermal cycling may include using a thermal cycler, isothermal amplification, thermal convention, infrared mediated thermal cycling, or helicase dependent amplification, for example.

According to various embodiments, detection of a target may be, but is not limited to, fluorescence detection, detection of positive or negative ions, pH detection, voltage detection, or current detection, alone or in combination, for example.

One should appreciate that the disclosed techniques provide many advantageous technical effects including automated methods for genotyping one or more rare genetic variants in a biological sample using an analyte detection (e.g., a PCR) apparatus. The techniques described herein employ logic to automate various processes, including processes currently performed using manual human effort. Further, the disclosed techniques have been designed to support data accuracy and allow for processing data algorithms and complex permutations on a scale and speed that cannot be achieved using manual human effort.

It should also be appreciated that the following specification is not intended as an extensive overview, and as such, concepts may be simplified in the interests of clarity and brevity.

All publications, patents, and patent applications mentioned in this specification are herein incorporated by reference to the same extent as if each individual publication, patent, or patent application was specifically and individually indicated to be incorporated by reference.

Embodiments of the invention discussed herein utilize microarray analysis. A microarray is a substrate to which are attached millions of single-stranded DNA complementary to the DNA to be detected. Microarrays may come in several flavors; e.g., where the substrate is a small plastic or glass slide like a microscope slide or a plastic bead. There can be from a few thousand to 1 million probes each consisting of thousands of single strands with the same sequence. The probes are attached to the substrate of the microarray. Microarrays are generally species or genotype specific, although the same array can sometimes be used for closely related species, e.g. humans and chimpanzees. In microarray analysis, it is generally needed to know what sequences are to be investigated because microarrays need known sequences to be used as probes. Data capture generally cannot occur without a probe. In addition, genetic variation affects how intensely the complemental DNA hybridizes.

Probes or probe sets need to be chosen to provide sufficient sensitivity (i.e., the ability to detect the rarely expressed transcripts in a complex background), and specificity (i.e., the ability to distinguish measures among transcripts with high sequence similarity), as well as high coverage (i.e., the ability to include all relevant transcripts to the experiment). It is desired to avoid sequences that are ambiguous (i.e., hybridize to multiple transcripts) or highly similar to non-target transcripts (i.e., cross-hybridization). Additionally, redundancy (i.e., several probes or probe sets targeting the same transcripts) can increase the accuracy of measures but it can at the same time reduce the coverage. Furthermore, the successful application also requires correct and up-to-date annotation (i.e., the association of probes with target transcripts) of the probes or probe sets.

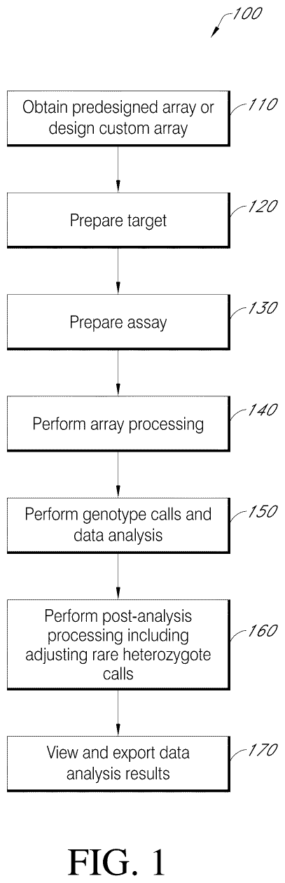

FIG. 1 is a flowchart illustrating a method 100 for genotyping rare genetic variants according to various embodiments. In step 110, a microarray such as a custom array is designed, or a predesigned array is obtained for the genotyping analysis. In step 120, the target (sample under investigation) is prepared. In step 130, the assay is prepared. The array is processed in step 140.

In one embodiment of the invention, total genomic DNA is amplified and randomly fragmented from a sample. These fragments are purified, re-suspended, and hybridized to complementary probes. Following hybridization, the bound target is washed under stringent conditions to remove non-specific background. Each allele in the target DNA is queried via a multi-color ligation event. After ligation, the arrays are stained and imaged. The intensity of the image of each allele (A or B) of the target is expected to be roughly proportional to amount of each allele in the DNA sample.

A dye intensity for each probe is summarized by a scanning microscope which essentially takes a photo of the microarray at the wavelength of the label. The raw data is the intensity of reflectance of the label at each pixel. This intensity is expected to be proportional to the amount of material in the labeled sample.

Fundamental data generated during the array processing step 140 may include digitized photo(s) of the array giving the label intensity.

Genotype calls and data analysis is performed at step 150. In some embodiments of the invention, genotyping analysis may be performed by genotyping analysis software manufactured by Thermo Fisher Scientific, such as Applied Biosystems™ Array Power Tools (APT), e.g., APT version 2.11.3 or other versions, or Applied Biosystems™ Axiom™ Analysis Suite Software (AxAS), e.g., AxAS version 5.1, 5.1.1, or other versions. In some embodiments of the invention, custom and catalog genotyping arrays manufactured by Applied Biosystems™ under the Axiom™ name may offer highly accurate genotyping for custom and rare variants. The use of genotyping arrays for extremely rare variants, for example, those variants with a Minor Allele Frequency (MAF), below 0.01%, can be challenging. Genotyping algorithms that are tailored to rare or very rare variants are therefore needed to improve the accuracy of calls of such variants. Such algorithms to adjust rare heterozygote calls may be included in post analysis processing step 160, and/or utilized in conjunction with genotyping analysis software or algorithms such as Applied Biosystems™ Array Power Tools (APT), e.g., APT version 2.11.3 or other versions, or Applied Biosystems™ Axiom™ Analysis Suite Software (AxAS), e.g., AxAS version 5.1, 5.1.1, or other versions, including Axiom GT1 genotyping. Rare Heterozygote (Het) Adjusted Genotyping algorithms discussed herein in relation to embodiments of the present invention may be used with other genotyping analysis software or algorithms known in the art from other manufacturers. Such software will allow a user to view and export data analysis results at step 170.

While sequencing can still be prohibitive for large studies, commercially available genotyping arrays targeting rare variants prove to be a reasonable alternative. A technical challenge of array-based methods is the task of deriving genotype classes (homozygous or heterozygous) by clustering intensity data points. The performance of clustering tools for common polymorphisms is well established, while their performance when conducted with a large proportion of rare variants (where data points are sparse for genotypes containing the rare allele) is less known. In one embodiment of the present invention, genotyping is done using a clustering algorithm with very high accuracy in the Axiom GT1 genotyping process.

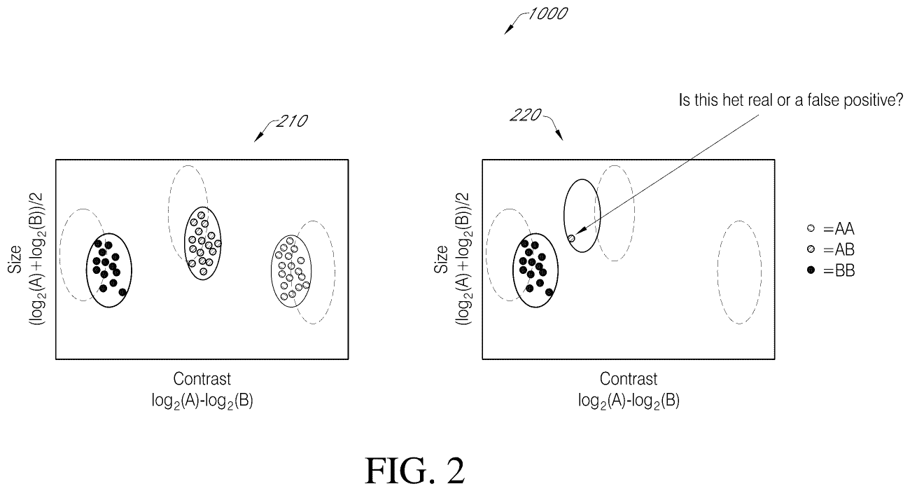

FIG. 2 illustrates genotype cluster plots according to various embodiments. In one embodiment of the present invention, AxiomGT1 is a tuned version of the BRLMM-P (Affymetrix, 2007) clustering algorithm, which adapts pre-positioned genotype cluster locations (priors) to the sample data and calculates three posterior cluster locations. Genotype cluster locations for data with A and B alleles are defined by 2D means and variances for AA, AB, and BB genotype cluster distributions. Priors can be generic, meaning the same prepositioned location is provided for every SNP, or SNP specific, meaning the different pre-positioned locations are provided on a SNP-by-SNP basis. Clustering is carried out in two dimensions as shown in genotyping cluster plots 210 and 220. The X dimension is called “contrast” and the Y dimension is called “size”. They are log-linear combinations of the two allele signal intensities. For alleles A and B, contrast is log 2(A/B) and size is (log 2(A)+log 2(B))/2. The genotype cluster plots produced by Axiom Analysis Suite and Ps_Visualization label the axes either with the size/contrast pair or with the formulas for size and contrast. Genotype calls are made by identifying the genotype intensity cluster (AA, AB, or BB) each sample is most likely to belong to. The samples are colored and shaped by these genotype calls in the cluster plots: BB calls are red circles, AB calls are green circles, and AA calls are yellow circles. Note that is it possible to color the data according to other sample attributes, with different options in Ps_Visualization and the SNP Cluster Graph. Arrays used in embodiments of the present invention call SNP genotypes as AA, AB, or BB using a clustering algorithm with very high accuracy that works well when multiple clusters are well-populated, such as in plot 210, but SNP genotype calling may be less accurate when only one cluster is well-populated, as shown in plot 220, where a false positive het call may be more likely for the AB genotype call where there is only one AB genotype call shown.

Thus, in one embodiment of the invention, the Axiom Rare Heterozygous Adjusted (RHA) Genotyping algorithm optimizes Axiom GT1 genotyping for very rare variants. While the location and shape of the heterozygous (het) cluster provides powerful evidence for het calls for common variants, rare variants often have a single sample in the het cluster, making the call less robust. In-depth analysis of the distribution of replicate probe signals on Axiom microarrays reveal this distribution can be used to significantly improve the accuracy of heterozygous calls.

In one embodiment of the invention, the RHA algorithm is executed after an initial genotyping algorithm (e.g., Axiom GT1), to determine which predictions of rare heterozygous (Het) genotypes are likely to be false. The genotypes of false Het predictions, as determined by RHA are then set to “No Call”. A main insight of the algorithm, after examining many hundreds of different array results, is that most false Het predictions occur due to a small number of higher or lower than expected intensities on the microarray surface. These unexpected intensities may cause a homozygous sample's summarized probeset intensity to be positioned away from the major homozygous genotype cluster in “signal contrast” vs. “signal size” (clustering) space and into a location that could be consistent with a heterozygous genotype cluster, thereby causing the AxiomGT1 genotyping algorithm to falsely predict a Het.

In one embodiment of the invention, an initial genotyping algorithm (e.g., Axiom GT1) is used to genotype an exemplary health resource such as the UK Biobank, which follows the health and well-being of 500,000 people, all genotyped on Thermo Fisher Scientific's Applied Biosystems™ Axiom™ microarrays. Whole exome sequencing (WES) for a subset of participants was also made available, for example, an initial WES release for the UK Biobank contained data for about 50,000 participants. In one embodiment of the invention, the Axiom GT1 algorithm used to genotype the UK Biobank already incorporates detection and elimination of unexpected intensities into its genotype calling but does not use higher stringency for low minor allele frequency (MAF) variants. However, genotyping variants for which Hets are rare in the population is especially challenging because false Het predictions are more likely to occur in cases where the number of true Hets is too low (or zero in the most extreme case) to correctly anchor the Het cluster. The RHA algorithm improves rare Het prediction by incorporating additional information in the test for unexpected intensities. These include: (1) the intensity differences of replicated probe sequences on the array for the putative Het sample and (2) the position of the putative Het signal relative to the signal distribution of homozygous samples. In one embodiment of the invention, the RHA algorithm eliminates (sets to NoCall) the majority of false positive heterozygous calls, while leaving true positive heterozygous calls virtually untouched.

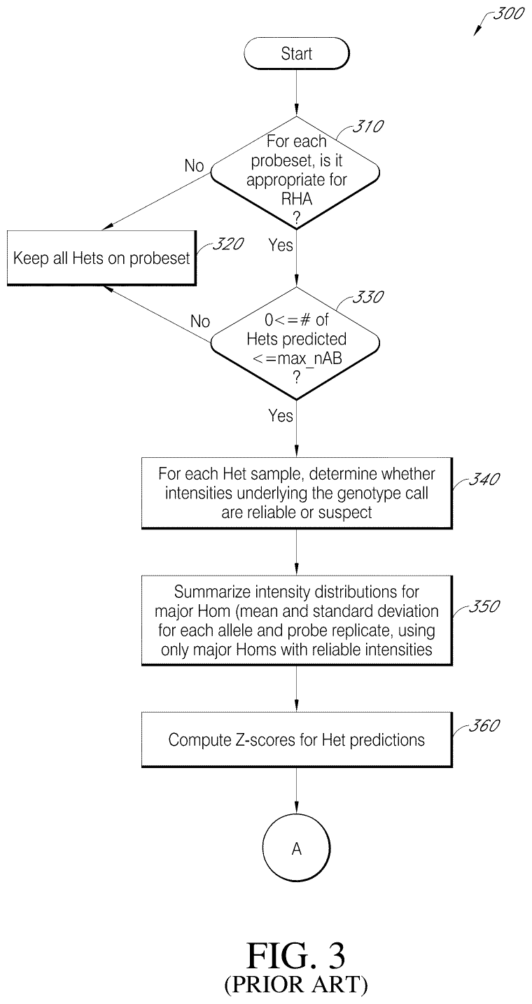

RHA as used in one embodiment of the invention is summarized in the exemplary flowchart 300 of FIG. 3. The RHA algorithm is applied at step 310 only to probesets with two replicate probes and a small number of Hets. Hets on probesets not meeting these requirements are retained at step 320.

In the UK Biobank release used in one embodiment of the invention, samples were genotyped in batches of about 5,000 samples and RHA is applied only if the number of Het predictions in the particular batch is between 1 and max_nAB, as shown in step 330. In some embodiments of the invention, the RHA algorithm examines heterozygous calls from probesets that have two replicates on the array and which have a max_nAB of three or four, or less heterozygous (Het) calls in the batch. Max_nAB is a parameter of the algorithm. Several metrics the algorithm uses are also applicable to probesets with one replicate.

At step 340, all Het calls meeting the criteria for very rare variants at step 330 are tested for patterns of “unexpected intensities” which are defined as an inconsistency in intensities between the two replicate probe sequences for the Het. If there are no unexpected intensities, the prediction is deemed to be true at step 320, and the Het genotype call is retained.

Otherwise, the prediction undergoes further inspection to determine whether the individual probe intensities are “Het-like”. All Het calls for rare variants are tested for patterns of “unexpected intensities” which are defined as an inconsistency in intensities between the two replicate probe sequences for the Het. If there are no unexpected intensities, the prediction is deemed to be true, and the Het genotype call is retained.

In one embodiment of the invention, the AxiomGT1 genotyping algorithm clusters samples in “signal contrast” vs. “signal size” (clustering) space to obtain genotypes, after summarizing the intensities for the various replicates of the same probe. Different replicates of probe sequences for the same probeset may have a slightly different intensity distribution as a result of their locations on the array. Minor non-uniformities on the array surface can cause a probe's intensity to be lower or higher than it should be.

With RHA as an added enhancement to an initial genotyping algorithm such as Axiom GT1, a Het prediction is marked as suspect and likely requiring further inspection at step 340, if it is a rare Het (Het cluster size<=max_nAB) and one of two criteria for an unexpected intensity is met:
a) One of the probeset's probe replicates was eliminated by the Axiom GT1 algorithm because of an unexpected intensity. For such probes, the genotyping algorithm uses just one of the replicates to genotype the sample. However, on occasion the single remaining intensity may cause a shift in the position of a homozygous sample in clustering space that is sufficiently large and in an appropriate direction for the genotyping algorithm to call a Het.
b) The difference between intensities measured on different probe replicates is higher than expected. To predict and classify probe intensities as unexpected, a SCI (Sequence Contrast Index) metric is defined. SCI is calculated separately for the A and B alleles, using I1 and I2, the normalized intensities for the two probe replicates and can easily be generalized for more replicates:

$$SCI ( A ) = abs ( I 1 ( A ) - I 2 ( A ) ) I 1 ( A ) + I 2 ( A ) ; \qquad ( 1 )$$

$$SCI ( B ) = abs ( I 1 ( B ) - I 2 ( B ) ) I 1 ( B ) + I 2 ( B ) \qquad ( 2 )$$

SCI equals 0 for identical intensities and approaches 1 as the difference between intensities grows. Because both replicates of a probe are unlikely to experience the same type of unexpected intensity at the same time, a low SCI indicates that intensities measured on both probe replicates are reliable. If SCI is high for at least one of the alleles, the Het prediction is marked for further inspection. A cutoff value for SCI was empirically derived and has been shown to be applicable for different arrays and batch sizes.

Otherwise, or if the probeset has a single replicate and SCI cannot be computed, the prediction undergoes further inspection to determine whether the individual probe intensities are “Het-like”. Not all Het predictions with unexpected underlying intensities are false. To minimize loss of true positive Hets, suspect Het predictions may undergo a further examination. Ideally, the intensities driving the Het prediction are compared to a distribution of expected intensities for a Het genotype, as is done for higher minor allele frequency (MAF) variants. However, this requires many examples of true Hets for the given variant, which are generally not available for low MAF variants.

Instead, the predicted Het intensities to the distribution of intensities for the major Hom are compared. A major Hom genotype is comprised of two copies of the major allele and zero copies of the minor allele, whereas a Het genotype is comprised of one copy of each allele. These differences should be reflected in the intensity measurements of each of the two alleles and can therefore be used to differentiate the two genotypes. The advantage of this approach is that for low minor allele frequencies the vast majority of genotypes are major Homs, giving us plenty of examples to characterize the major Hom intensity distribution for a given replicate as shown in step 350.

While the intensity distribution differs for different probesets, and sometimes for different replicates, the range of positions relative to the mean and variance of this distribution that is appropriate for a Het is generally universal for all probesets. The intensities underlying the Het prediction into Z-scores are therefore translated at step 360.

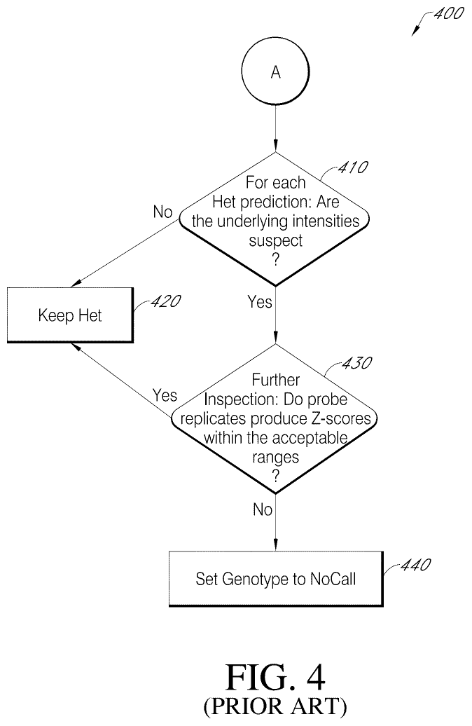

In FIG. 4, the analysis used in embodiments of the present invention continues on exemplary flowchart 400. Suspect probe intensities determined at step 410 may be compared relative to the appropriate major Hom intensity distribution and checked to determine whether these Z-scores fall in the “acceptable” range as shown in step 430 (different ranges are appropriate depending on whether the intensity is measured for the major or minor allele). In this way, it is possible to check for each probe-replicate whether the intensities measured on it are uniformly Het-like.

If the prediction passes this second inspection, the Het genotype call is retained in step 420, otherwise the genotype of the probeset on the sample is set to “No Call” (NC) at step 440. The hets are selectively adjusted to “No Calls”, when the distribution of replicate probe signals for the sample and probeset in question suggest that the call is a false positive. The number of het calls that are examined and set to No Call depends on the array, and more specifically on the number of rare variants on the array. In one embodiment of the present invention, typically less than a hundred calls are adjusted in a batch of 96 samples genotyped on a high-density array. In general, the rare het adjustment affects on average less than one het per sample.

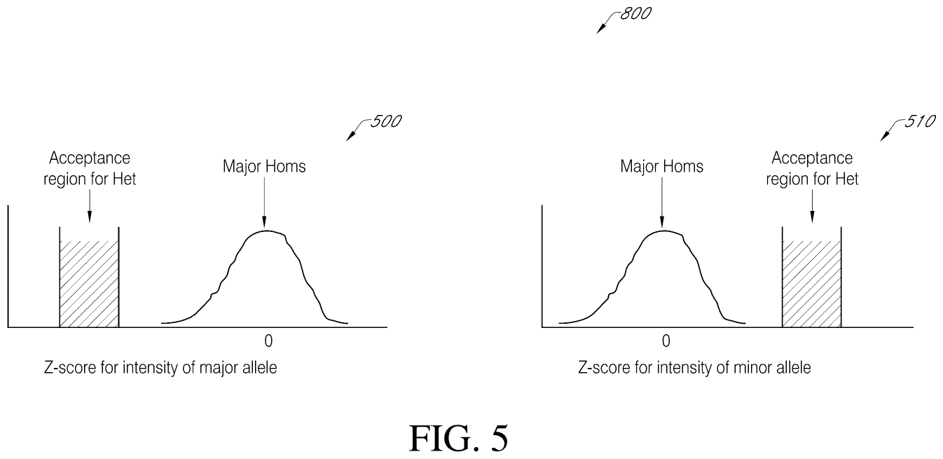

FIG. 5 shows exemplary illustrations of acceptable ranges for Het intensity Z-scores relative to the distribution of intensities from major Hom samples used in embodiments of the present invention. Z-scores for the intensities of predicted Hets are calculated, based on summary statistics (mean and standard deviation) of the distribution of corresponding intensities for major Homs on the particular probeset, using the following formula:

$$Zscore = \frac{[ Intensity - Mean ( HomIntensity ) ]}{Std ( HomIntensity )}$$

In one embodiment of the invention, only major Hom genotypes with reliable underlying intensities are used to calculate these summary statistics. In addition, Z-scores are computed only when the major allele can be determined with confidence (at least 90% of samples are major Hom) and number of high-quality major Hom genotypes is sufficiently high (at least 30). Finally, if it is not possible to obtain such summary statistics for major Homs, all Hets with underlying aberrant intensities are set to ‘No Call’.

FIG. 5 at 500 shows an exemplary acceptance region for the Z-score of the major allele intensity. Since major Hom's have two copies of the major allele, whereas a Het has only one copy of this allele the Z-score is expected to be negative. However, intensity values and their associated Z-scores that are too low may be indicative of a probe that isn't working and are thus not accepted. Graph 510 of FIG. 5 shows an exemplary acceptance region for the Z-score of the minor allele intensity. Major Hom's have zero copies of the minor allele, and their intensity on this allele is thus the background noise, whereas a Het has one copy of this allele. Thus, it is expected that the Z-score for the minor allele intensity would be positive. On the other hand, a Z-score that is too high could indicate that the intensity measured on the probe is aberrantly high.

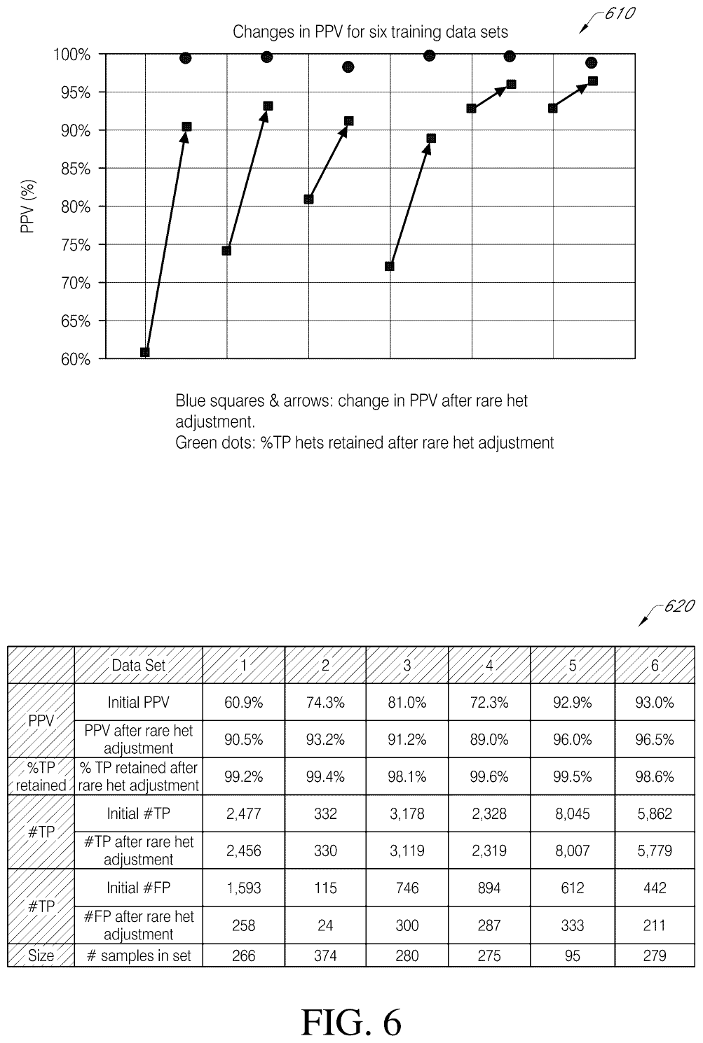

Embodiments of the invention may be designed and trained on various independent datasets. One important metric for rare het calls is the Positive Predictive Value (PPV) of such calls. PPV is the fraction of correct het calls out of all het calls, or TruePositive/(TruePositive+False Positive). For example, FIG. 6, in graph 610, shows the number of samples in each dataset, and the Positive Predictive Value (PPV) for six representative data sets before and after the application of RHA genotyping to clusters with a single het, all genotyped with 1000 Genome samples with published genotypes, where the application of RHA clearly increases the PPV to above 90% for all six representative datasets as shown in the blue squares and arrows of graph 610. Table 620 shows the data for the six representative datasets, including initial PPV, PPV after RHA, and % TP where % TP is the percent of true Hets retained after RHA. The % TP is an important metric that is the number of True Positive calls retained by the RHA algorithm. Graph 610 in FIG. 6, in the green dots, further shows that on average over 99% of the true positive calls are retained after RHA application. Table 620 further shows other raw data such as the initial number of true positives, #TP and the #TP after RHA, and the initial number of false positive het calls, #FP, and the #FP after RHA.

Similar results were obtained when embodiments of the invention were applied to UK Biobank data. In one embodiment of the invention, truth data may be taken from whole-exome sequencing (WES) data from the UK Biobank for 200K individuals. The UK Biobank Data is a health resource that follows the health and well-being of 500,000 human participants. The participants' genetic data is genotyped on Thermo Fisher Scientific's Applied Biosystems Axiom UKBiobank and UKBileve microarrays.

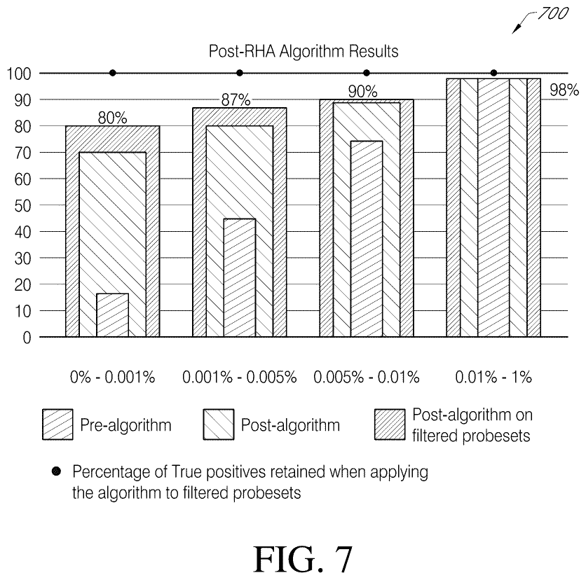

FIG. 7 illustrates a graph showing results of an RHA algorithm used in one embodiment of the invention on UK Biobank data. Before describing the verification results on the UK Biobank data, Minor Allele Frequency (MAF) bins are defined, as described in Weedon et al., (2019) Assessing the analytical validity of SNP-chips for detecting very rare pathogenic variants: implications for direct-to-consumer genetic testing, https://www.biorxiv.org/content/10.1101/696799v2.

According to Weedon et al., UK Biobank probesets may be defined into Axiom computed MAF (AcMAF) bins, based on Axiom array genotypes in the UK Biobank data. The bins may be defined as shown in the table below. Note that Bins 1-3 have extremely low allele frequency. For example, Bin 1 has extremely low AcMAF with at most 9 of the nearly 500,000 individuals genotyped on the UK Biobank array having the minor allele. A variant in Bin 3 is expected to have less than 5-10 individuals with a het call among 50,000 individuals, so even Bin 3 has extremely low MAE. As shown in graph 700 in FIG. 7, RHA genotyping significantly improves PPV in all MAF ranges. PPV and sensitivity were calculated based on the exome sequencing data for ˜50,000 UK BioBank samples. Statistics were calculated on all heterozygous genotypes from probesets in the various computed MAF groups, applied on two-replicate probesets with a het cluster size of up to 4 within the respective batch. Probesets were filtered based on the rare het algorithm prediction. Bin 4 and Bin 5 have little to no rare variants, and are therefore not affected by the RHA genotyping algorithm. Bin 4 is shown in graph 700 of FIG. 7 for completeness, while Bin 5, with MAF greater than 1% is not shown as all values are virtually at 100% pre and post rare het adjustment.

| Bin | 1 | 2 | 3 | 4 | 5 |
|---|---|---|---|---|---|
| AcMAF | 0%-0.001% | 0.001%-0.005% | 0.005%-0.01% | 0.01%-1% | 1%-50% |

Validation or ground truth data (e.g., the known sequences to be investigated) can be obtained by whole exome sequencing or whole genome sequencing. Results of genotyping algorithms in one or more embodiments of the invention discussed herein may be compared to Exome sequencing data, available for ˜50,000 participants of the UK Biobank cohort.

Embodiments of the invention discussed herein utilize improved algorithms for genotype calls. Experimental data show that Axiom microarrays achieve excellent PPV for very rare variants, removing false het calls with high accuracy, while keeping true calls virtually intact. For very rare variants (below 0.01% MAF, e.g. less than 1 expected het in 5,000 individuals), Axiom Array genotypes were observed to give excellent performance _{oI2n} these markers, especially when curated with samples carrying the rare het to confirm performance.

When the variants are restricted to those probesets that produced at least one true positive het in the Exome sequencing data, a PPV of 98%, along with an average sensitivity of 96%, (data not shown) can be achieved. This superior performance was observed on the AcMAF Bin1, the most challenging bin.

In one embodiment of the invention, an additional level of quality control beyond RHA is needed to further reduce false Het calls for very rare variants, even though RHA already greatly reduces false Het calls. In such cases, a supervised machine learning classification model, such as a support vector machine prediction model, may be used. A support vector machine prediction model is described below with respect to embodiments of the invention, although other classification models known in the art, including but not limited to random forest or multilayer perceptron (MLP) classifiers, may be contemplated within the scope of the invention.

A support vector machine (SVM) predictor utilized in embodiments of the invention described herein starts with rare heterozygous adjustment (RHA) algorithm output. The SVM predictor thus provides additional quality control and predicts whether putative Hets that pass the RHA test (not set to NoCall) are actual Heterozygous (Het) genotypes (True Positive) or actual Homozygous (Hom) genotypes (False Positive). A support vector machine (SVM) predictor utilized in embodiments of the invention described herein starts with rare heterozygous adjustment (RHA) algorithm output.

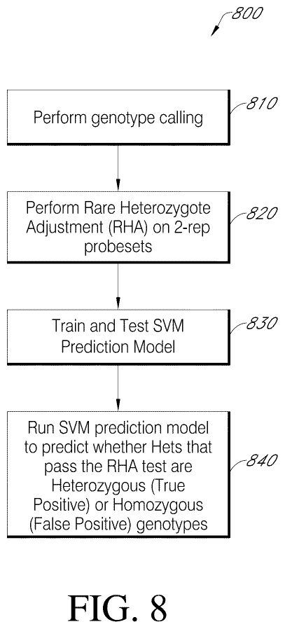

FIG. 8 is a flow chart showing a method 800 of genotyping rare variants including a support vector machine analysis according to some embodiments of the present invention. In step 810, genotype calling is performed as described above, such as using the Axiom GT1 genotyping algorithm. In step 820, rare heterozygote adjustment (RHA) genotyping is performed as described above on 2-rep probesets. In one embodiment of the invention, step 820 may not be performed if rare Het genotypes identified from Axiom GT1 genotyping produced by 1-rep probesets are being evaluated.

In step 830, a support vector machine (SVM) prediction model may be trained and tested. For the SVM prediction model, in one embodiment of the invention, truth data may be taken from the second release of whole-exome sequencing (WES) data from the UK Biobank form 200K individuals. In the truth data, 8988 were true positives (TP), where the Axiom GT1 with RHA calls Het and the exome truth call is also Het. In the truth data, 2764 were false positives (FP), where the Axiom GT1 with RHA algorithm calls Het, but the WES truth call is homozygous. The truth data is then divided as follows: 80% of the truth data is utilized as training data for training the SVM predictor, and 20% of the truth data is utilized as test data. In one embodiment of the invention, five predictors may be utilized. The five predictors are as follows, four numeric and one Boolean (e.g., yes/no): minMinorSdDist (should be positive), maxMajorSdDist (should be negative), maxSCI (lower is better), Confidence Score (lower is better), and AlleleSpecific(AS) (e.g., yes AS or no AS; where nonAS is better). In step 840, the SVM prediction model is run to predict whether Hets that pass the RHA test are Heterozygous (True Positive) or Homozygous (False Positive) genotypes.

The five predictors are defined below as follows:
MaxSCI: SCI (Sequence Contrast Index) is calculated separately for the A and B alleles, using I₁ and I₂, the normalized intensities for the two probe replicates for the allele: maxSCI=Max (SCI(A), SCI(B), where

$$SCI ( A ) = \frac{abs ( I 1 ( A ) - I 2 ( A )}{[ I 1 ( A ) + I 2 ( A ) ]}$$

$$SCI ( B ) = \frac{abs ( I 1 ( B ) - I 2 ( B )}{[ I 1 ( B ) + I 2 ( B ) ]}$$

MaxSCI requires two or more replicates for a given probe, while the other predictors can be applied to probes with single replicates
minMinorSdDist, and maxMajorSdDist: Array intensities underlying the putative Het are translated into Z-scores (MinorSdDist, MajorSdDist) relative to the appropriate major Hom intensity distribution. Specifically: for each replicated probe sequence, i=1 to 2,
   MinorSdDist_i=[IntensityOfThePutativeHet_i−Mean(HomIntensity_i)]/Std(HomIntensity_i) in the Axiom channel for the Minor Homozygous allele;
   MajorSdDist_i=[IntensityOfThePutativeHet_i−Mean(HomIntensity_i)]/Std(HomIntensity_i) in the Axiom channel for the Major Homozygous allele;
   minMinorSdDist=min(MinorSdDist_i);
   maxMajorSdDist=max(MajorSdDist_i);
Confidence Score: The confidence score for an Axiom genotype call produced by the Axiom™ GT1 algorithm or other suitable genotyping algorithm as described herein.
AlleleSpecific (AS) Y/N): An indicator of whether the probeset for the rare variant was using an AlleleSpecific probeset design or a Non-AlleleSpecific probeset design.

In one embodiment of the invention, the support vector machine prediction model is run using the R kernel. Support Vector Machine (or SVM) is a machine learning technique used for classification tasks. Briefly, SVM works by identifying the optimal decision boundary that separates data points from different groups (or classes), and then predicts the class of new observations based on this separation boundary. Depending on the situations, the different groups might be separable by a linear straight line or by a non-linear boundary line. Support vector machine methods can handle both linear and non-linear class boundaries. It can be used for both two-class and multi-class classification problems. In real life data, the separation boundary is generally nonlinear. Technically, the SVM algorithm performs a non-linear classification using a kernel transformation. The most commonly used kernel transformations are polynomial kernel and radial kernel. The Support Vector Machine (or SVM) is a useful classification technique. To build a non-linear SVM classifier, either polynomial kernel or radial kernel function, or other appropriate kernel transformation techniques may be used.

The package automatically chooses the optimal values for the model tuning parameters, where optimal is defined as values that maximize the model accuracy. To build a non-linear SVM classifier, as discussed above, one can use either polynomial kernel or radial kernel function. Again, the caret package can be used to easily compute the polynomial and the radial SVM non-linear models.

In one embodiment of the invention, an SVM classifier is built using the caret R package. In one embodiment of the invention, R packages may be loaded, such as tidyverse to assist in facilitating data manipulation and visualization, and caret to assist in facilitating machine learning workflow. In one embodiment of the invention, the UK BioBank data is used as training data. Variables may be normalized to make their scale comparable. This may be automatically done before building the SVM classifier by setting the option preProcess=c(“center”,“scale”). The SVM radial basis function classifier may be fitted as follows. The package automatically chooses the optimal values for the model tuning parameters, where optimal is defined as values that maximize the model accuracy.

```
svm_Radial <- train(Concordance_with_200Kexome ~., data =
train_ukbb_ss,
method = “svmRadial”,
  trControl=trctrl,
  preProcess = c(“center”, “scale”),
    tuneLength = 10)
trctrl <- trainControl(method = “repeatedcv”, number = 10, repeats = 3,
savePredictions = TRUE)
```

The SVM prediction model may be viewed as follows: Support Vector Machines with Radial Basis Function Kernel
9401 samples
   5 predictor
   2 classes: ‘0’, ‘1’
Pre-processing: centered (5), scaled (5)
Resampling: Cross-Validated (10 fold, repeated 3 times)
Summary of sample sizes: 8461, 8461, 8461, 8461, 8461, 8461, . . .
Resampling results across tuning parameters:
C Accuracy Kappa
   0.25 0.9286606 0.7928662
   0.50 0.9288734 0.7940837
   1.00 0.9294050 0.7961062
   2.00 0.9293694 0.7962098
   4.00 09292277 0.7959212
   8.00 0.9277740 0.7918608
   16.00 0.9268166 0.7893567
   32.00 0.9262140 0.7877711
   64.00 0.9251860 0.7849598
   128.00 0.9239804 0.7818771
Tuning parameter ‘sigma’ was held constant at a value of 0.6126916
Accuracy was used to select the optimal model using the largest value.
The final values used for the model were sigma=0.6126916 and C=1.

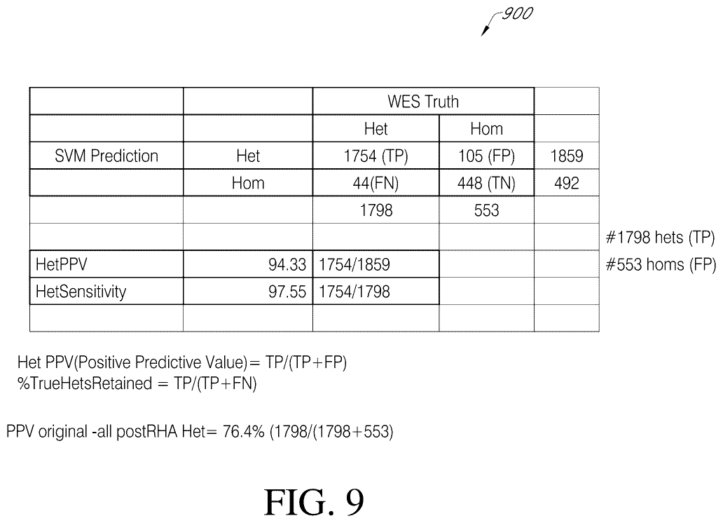

FIG. 9 shows a table 900 of test results on UK Biobank data according to one embodiment of the invention. In one embodiment of the invention, truth data was taken from the second release of whole-exome sequencing (WES) data from the UK Biobank for 200,000 individuals. The test data was done on post RHA probesets. Of the WES data, 8988 were True Positives (TPs), where the Axiom GT1 algorithm calls Het and the exome truth call is Het. Of the WES data, 2764 were False Positives (FPs), where the Axiom GT1 algorithm calls het, where the exome truth data is Homozygous. As discussed above, the truth data is then divided as follows: 80% of the truth data is utilized as training data for training the SVM predictor, and 20% of the truth data is utilized as test data. Thus, the WES truth data used as test data is for 2351 individuals, with 1798 Heterozygous calls (True Positive), and 553 Homozygous calls (False Positive). Overall, the SVM predictor model increases Positive Predictive Value (PPV) by 18% (improvement from 76.4% for the post Axiom GT1 and RHA, to 94.33% with the SVM predictor model).

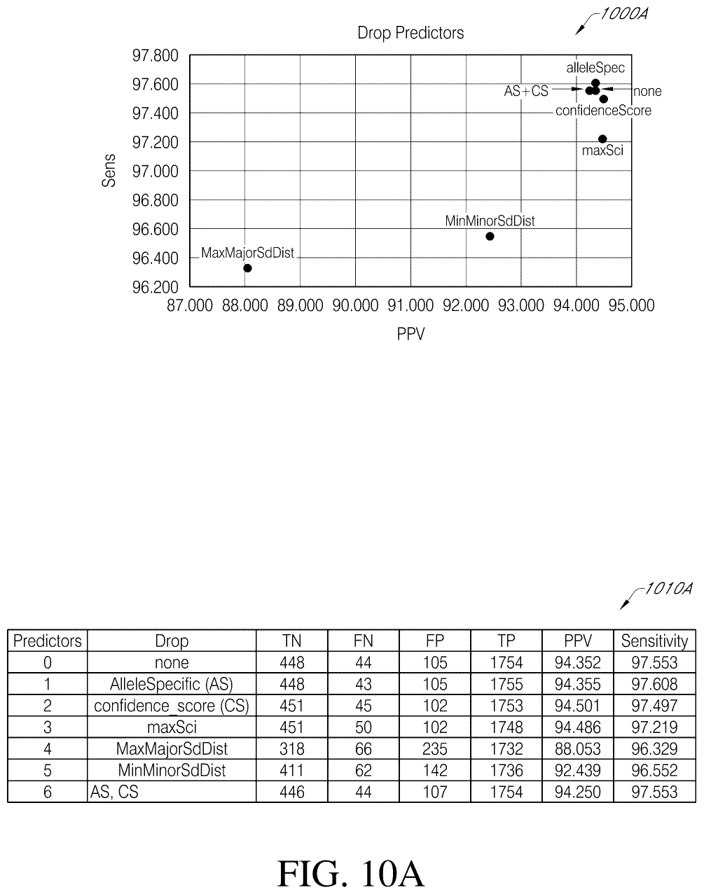

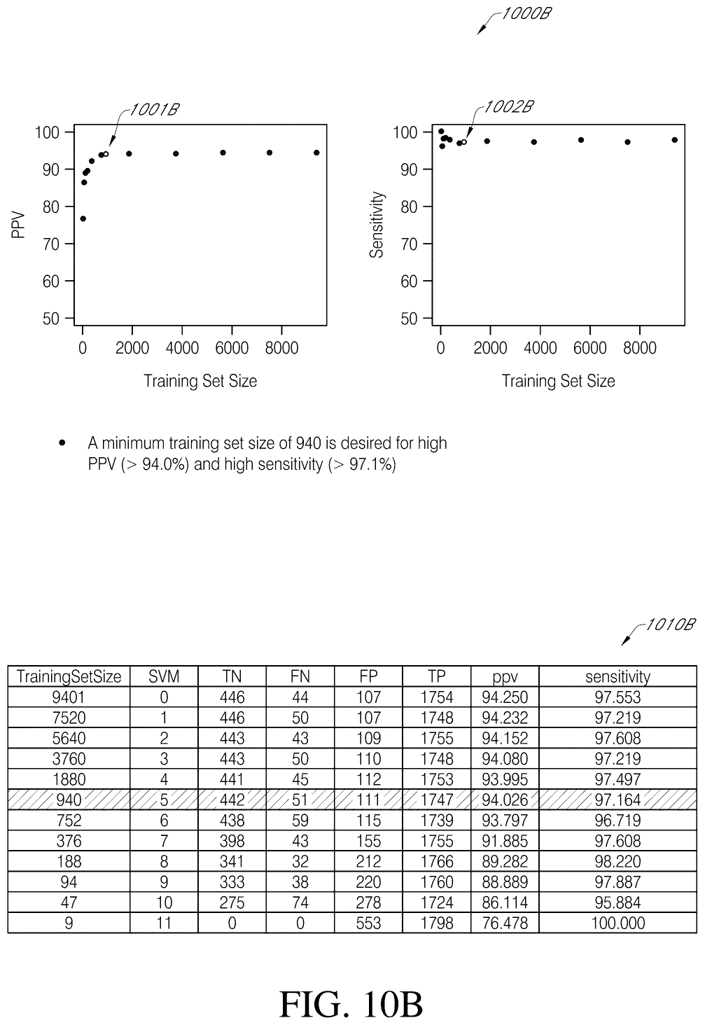

Thus, in some embodiments of the invention as shown in FIG. 10A, applying the 5 predictor SVM predictor model for rare post-RHA Het genotypes produced by “2 rep” probesets as described above increases the post-RHA PPV by 18% (76.4% to 94.33% while dropping the % True Hets Retained by only 2.5% (100% to 97.55%). In one embodiment of the invention, two predictors, ConfidenceScore and AlleleSpecific, can be dropped with essentially no effect on PPV and % TrueHetsRetained compared to the original 5-predictor model. Thus, in one embodiment of the invention, only three predictors are needed to run the SVM prediction model: maxSci, MaxMajorSdDist, and MinMinorSdDist. Dropping any of these three predictors maxSci, MaxMajorSdDist and MinMinorSdDist decreases PPV and Sensitivity as shown in table 1000A and graph 1010A.

In one embodiment of the invention, as discussed above with respect to UK BioBank Data, the input for the support vector machine prediction model comprises data points corresponding to at least 9400 training set heterozygous genotype calls. Of course, in other embodiments of the invention, data sources having different sizes of training sets of heterozygous genotype calls may be used as input for the support vector machine prediction model. As shown in FIG. 10B, embodiments of the present invention may utilize an input size for the support vector machine prediction model comprising a set of data points having a minimum size of 940 training set heterozygous genotype calls for optimal results. For example, graphs 1000B (at data points 1001B and 1002B) and table 1010B of FIG. 10B show that a minimum training set size of 940 is needed for high PPV (>94.0%) and high sensitivity (>97.1%),

Other embodiments for creating the SVM with training data and running the SVM with test data are shown below:

```
  #create SVM with training data
  library(caret)
    trctrl <- trainControl(method = “repeatedcv”, number = 10, repeats =
3, savePredictions = TRUE,
```

classProbs = TRUE,summaryFunction =

```
twoClassSummary)
    set.seed(3233)
  svm_Radial <- train(Concordance_with_200Kexome ~., data =
train_ukbb_ss, method = “svmRadial”,
    trControl=trctrl,
    preProcess = c(“center”, “scale”),
    tuneLength = 10,
    metric = “ROC”)
    #run test data
    test_ukbb <-test_ukbb_ss[,c(−1)]
    test_pred <- predict(svm_Radial, newdata = test_ukbb, type =
    “prob”)
```

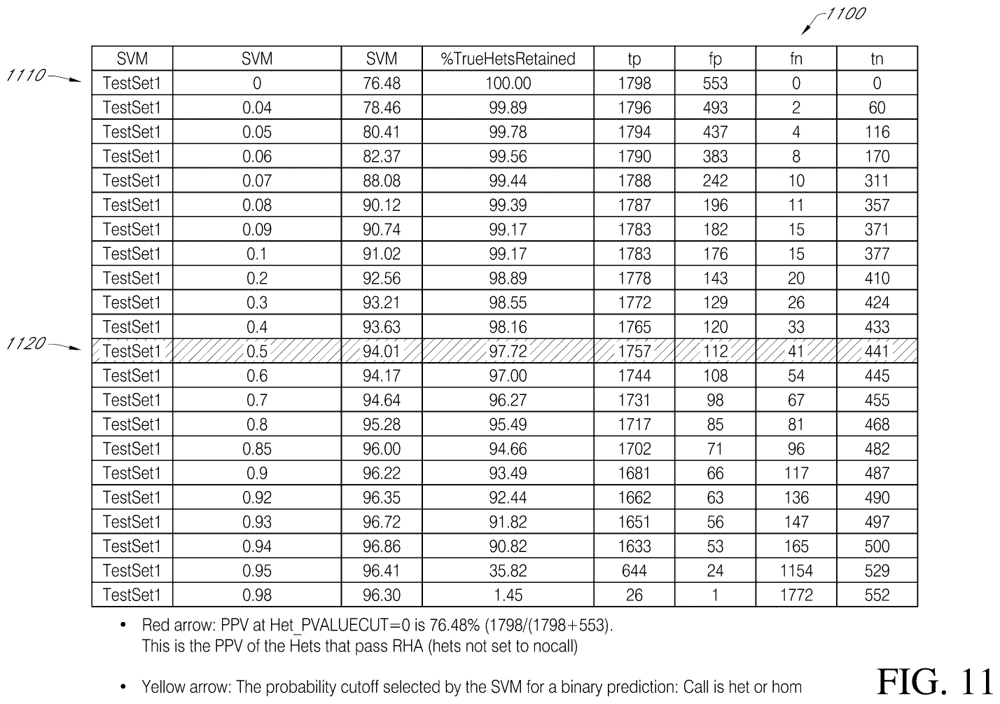

FIG. 11 illustrates a table of test results of 2-rep probesets showing the various probability cutoff values(Het_ProbabilityCUT) above which the SVM genotype is called Heterozygous (Het), and otherwise called Homozygous (Hom). TP (True Positive) is where the SVM genotype is predicted as Het, and the WES (truth data) genotype is Het. FP (False Positive) is where the SVM genotype is predicted as Het, and the WES genotype is Hom. FN (False Negative) is where the SVM genotype is Hom and the WES genotype is Het. TN (True Negative) is where the SVM genotype is Hom and the WES genotype is Hom. The “Red Arrow” 1110 is the PPV of the Hets that pass RHA (e.g., Hets not set to NoCall), where the Het_ProbabilityCUT is zero, meaning that the SVM prediction model will always make a Het call, so the PPV is the same as that of all Hets that pass RHA. The “Yellow Arrow” 1120 is the probability cutoff selected by the SVM for a binary prediction, where the SVM prediction model has an equal chance of predicting a Het or a Hom. In this case the % of True Het Calls retained (% HetsRetained=TP/(TP+FN)) is still high at 97.72%, while the Positive Predictive Value (HetPPV=TP/(TP+FP)) has gone up to 94.01% from 76.48%.

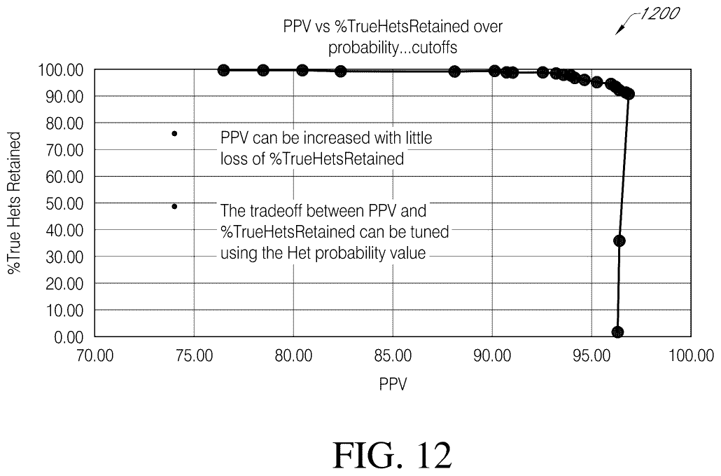

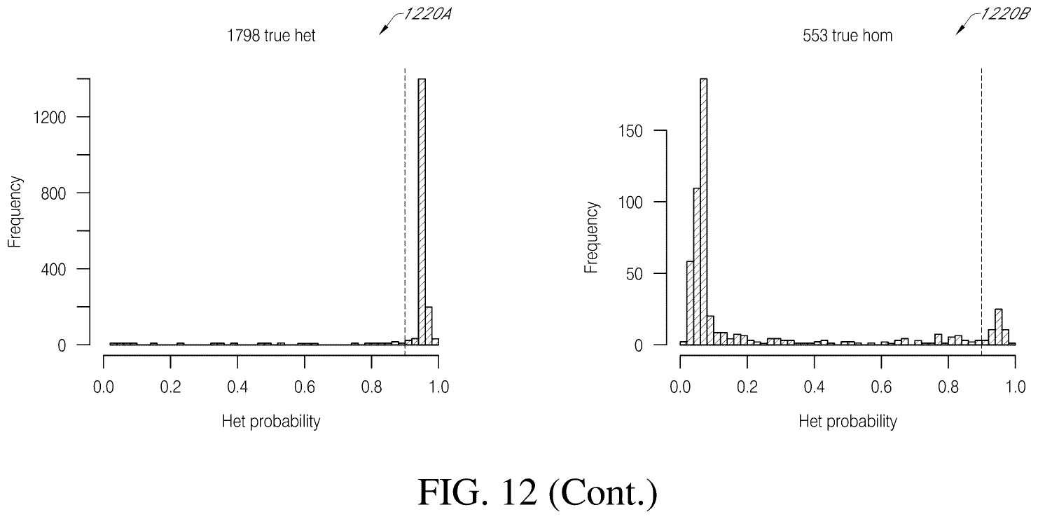

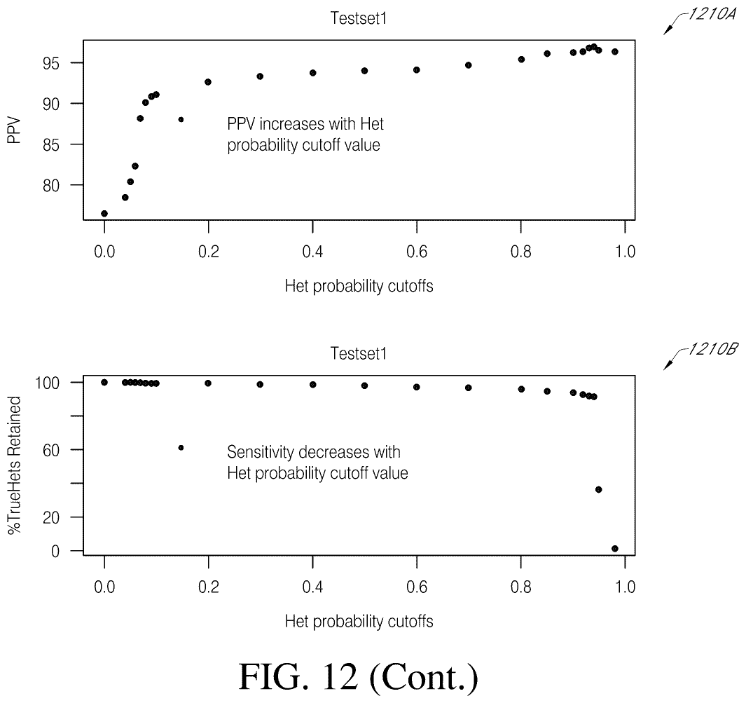

Graph 1200 in FIG. 12 show that PPV can be increased with little loss of % True Hets Retained, so that applying the SVM prediction model to the genotyping results of a microarray will increase the accuracy of the rare heterozygous genotype calling. The tradeoff between PPV and % of True Hets Retained can be tuned using the Het probability value as described above. Graph 1210A shows that PPV increases with Het probability cutoff value, and graph 1210B shows that % of true Hets retained decreases with Het probability cutoff value. This is because Het probabilities are high for true heterozygous calls as shown in graph 1220A and Het probabilities are low for true homozygous calls as shown in graph 1220B.

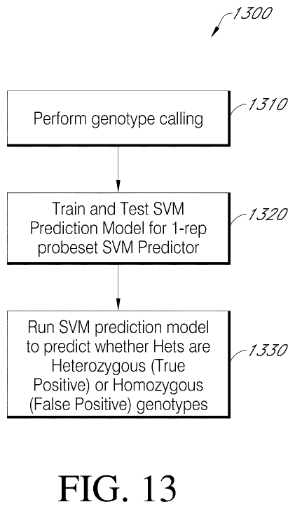

In some embodiments of the present invention, a support vector machine prediction model may be applied directly to genotyping data (e.g., genotyping data from Axiom GT1) that has not been separately adjusted for rare heterozygous calls. FIG. 13 shows an exemplary flowchart 1300 of such a process used in embodiments of the present invention. In one embodiment of the present invention, this SVM prediction model may be applied to rare Hets produced by 1-rep probesets. For example, for the UK Biobank microarray genotyping data, putative Het genotypes may be obtained from UK Biobank Het calls from Het genotype clusters with <=4 Het calls, and produced by probesets tiled with only one feature, called “1 rep” probesets. Note that in certain versions of the Axiom GT1 genotyping process as performed in step 1310, these Het calls are all done without a version of the Axiom GT1 RHA algorithm being applied, because Axiom GT1 RHA may not be applied to 1 rep probesets in some embodiments of the invention. It is also possible to develop a separate SVM model that may train on a single replicate in one embodiment of the invention.

In one embodiment of the present invention, truth data is taken from whole-exome sequencing (WES) data (second release) from UK Biobank for approximately 200,000 individuals. In one embodiment of the invention, 10% of 213596 het calls from WES truth data as described above is randomly sampled for training and testing the SVM prediction model in step 1320, although more or less data may be sampled as needed for computational performance. As in the previous SVM prediction models described herein, the WES truth data may be divided into 80% training data and 20% test data. In one embodiment of the present invention, the counts in the Testing set are: 3893 True Positives (TP), 379 False Positives (FP). Counts in the Training set are: 15490 TP and 1597 FP. In one embodiment of the present invention, three predictors may be applied: (1) MinorSdDist where the equation is as defined above, e.g., [IntensityOfthePutativeHet−Mean(HomIntensity)]/Std(HomIntensity); (2) MajorSdDist where the equation is as defined above, e.g., [(IntensityOfthePutativeHet−Mean(HomIntensity)]/Std(HomIntensity); and (3) nAB_inBatch, which is the number of hets in the Het genotype cluster (larger is better). Once the SVM prediction model is trained and tested, it may be run on experimental data at step 1330 to predict whether the rare Heterozygous calls generated by the genotyping assay are indeed Heterozygous (True Positive) or Homozygous (False Positive) genotypes.

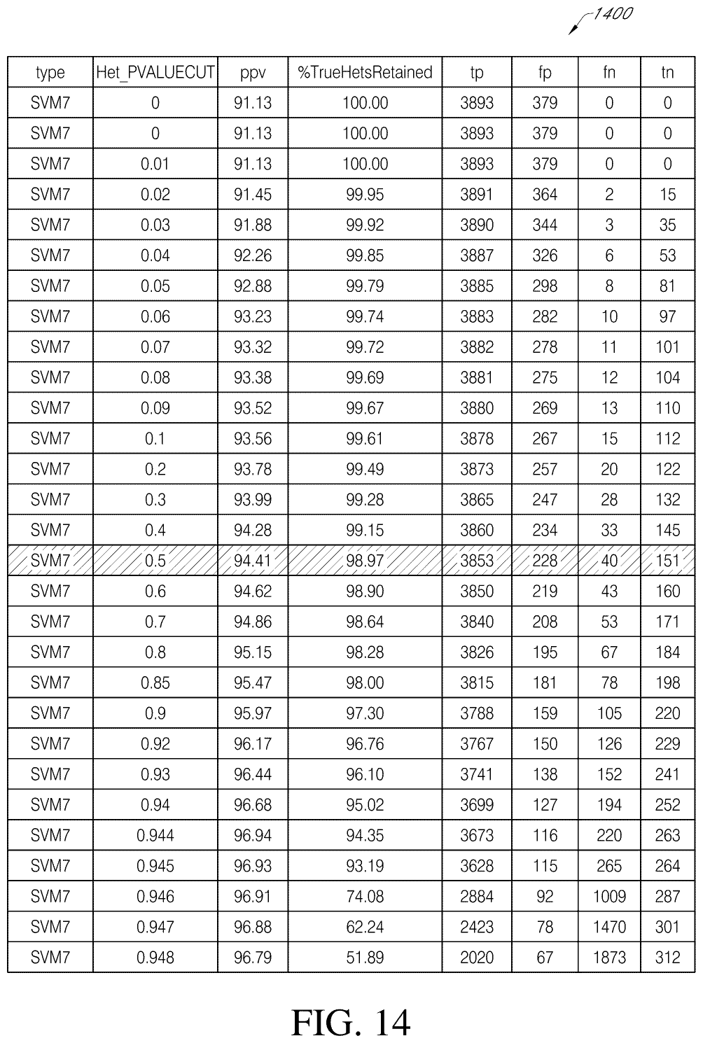

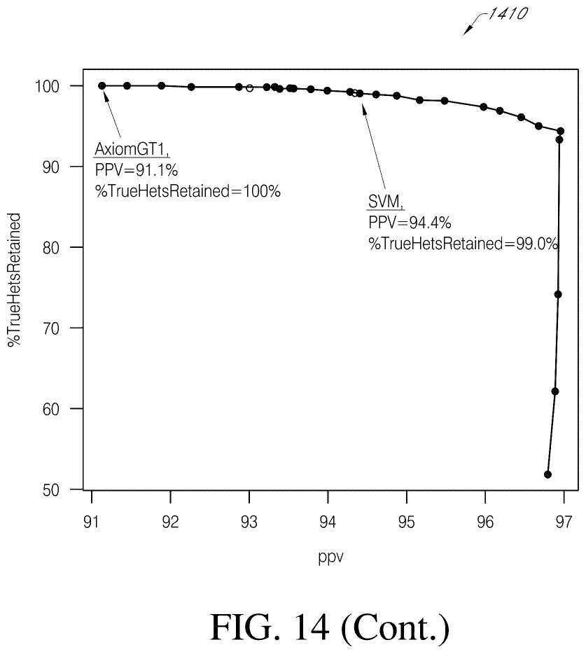

Table 1400 in FIG. 14 shows SVM test set metrics for 1 rep probesets, where different Het probability value cutoffs (Het_PVALUECUT) are listed for UK Biobank data used in one embodiment of the present invention. In one embodiment of the invention, the SVM prediction model produces a probability that the genotype is a Het for each Het call produced by the genotyping algorithm (e.g., Axiom GT1), with no separate rare Heterozygous adjustment step as described above. The rule for the Het_PVALUECUT is that if the Het probability is greater that the Het_PVALUECUT value, then the SVM call is Het, otherwise the SVM call is Homozygous. Graph 1410 illustrates a plot of PPV versus % True Hets Retained for different probability cutoffs as set forth in Table 1400. It is noted that the PPV for 1-rep probeset Hets start at a much higher value than 2-rep probeset Hets, at approximately 76% PPV for the 2-rep probesets versus 91.4%.

These test results occur because in the Axiom array design utilized in embodiments of the invention discussed herein, only probesets with high cluster resolution (e.g., 2-rep FLD >6) are selected to tile a 1-rep probeset, and FLD stands for Fisher's Linear Discriminant (FLD)=Min(i=aa,bb) {|Mab−Mi|/sd},

where Maa, Mbb, and Mab are the centers of the homozygous (aa, bb) and heterozygous (ab) clusters in the contrast dimension (X axis), and sd is the square root of the pooled variance across the three distributions.

Graph 1410 shows that the 1-rep SVM prediction model experimental results increases the PPV by 3%, from 91.4% produced by the standard Axiom GT1 algorithm to 94.4%, while only decreasing the % True Hets Retained by 1%.

In one embodiment of the present invention, the RHA algorithm output may determine whether a het call is “under blemish,” and the “under blemish” values are stored in an output file. The “blemishes” refers to physical imperfections that may be caused by blobs and scratches on the microarray surface that may result in differences in the intensity values measured on the microarray surface. Most het calls determined by RHA are not under blemish. In some embodiments of the invention, SVM predictor values may not be as accurate in determining whether a het call is a true het for het calls that are under blemish. For this reason, het calls under blemish are set to a probability for a value of zero in one embodiment of the invention, so this currently has a negligible effect on the percent true hets retained value. In one embodiment of the invention, it may be possible to toggle a setting or switch in the user interface, e.g., how the het calls under blemish are handled. In some embodiments of the invention, an SVM prediction model for hets under blemish may be developed using methods similar to those described above.

Systems, apparatus, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computers and interact via a network. The client-server relationship may be defined and controlled by computer programs running on the respective client and server computers. Examples of client computers can include desktop computers, workstations, portable computers, cellular smartphones, tablets, or other types of computing devices.

Systems, apparatus, and methods described herein may be implemented using a computer program product tangibly embodied in an information carrier, e.g., in a non-transitory machine-readable storage device, for execution by a programmable processor; and the method processes and steps described herein, including one or more of the steps of FIGS. 1, 3, 4, 8, and 13, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

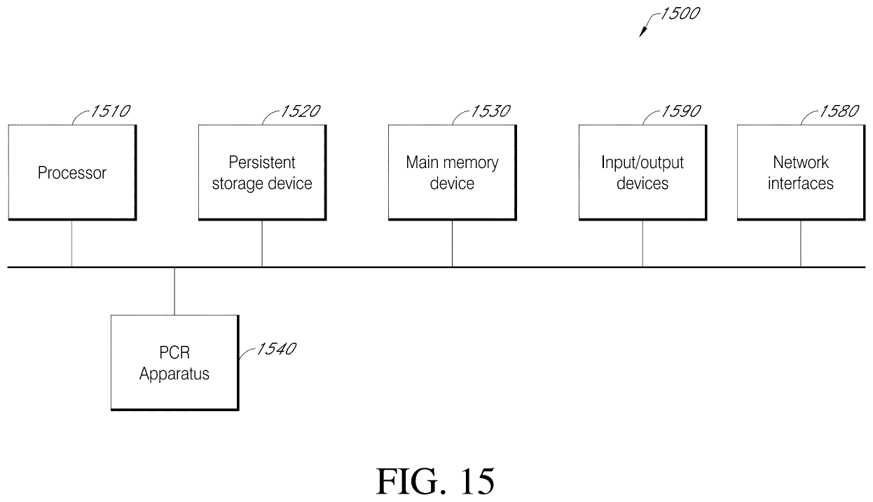

A high-level block diagram of an exemplary apparatus that may be used to implement systems, apparatus and methods described herein is illustrated in FIG. 15. Apparatus 1500 comprises a processor 1510 operatively coupled to a persistent storage device 1520 and a main memory device 1530. Processor 1510 controls the overall operation of apparatus 1500 by executing computer program instructions that define such operations. The computer program instructions may be stored in persistent storage device 1520, or other computer-readable medium, and loaded into main memory device 1530 when execution of the computer program instructions is desired. For example, processor 1510 may comprise one or more components of PCR apparatus 1540. Thus, the method steps of FIGS. 1, 3, 4, 8, and 13 can be defined by the computer program instructions stored in main memory device 1530 and/or persistent storage device 1520 and controlled by processor 1510 executing the computer program instructions. For example, the computer program instructions can be implemented as computer executable code programmed by one skilled in the art to perform an algorithm defined by the method steps of FIGS. 1, 3, 4, 8 and 13. Accordingly, by executing the computer program instructions, the processor 1510 executes an algorithm defined by the method steps of FIGS. 1, 3, 4, 8, and 13. Apparatus 1500 also includes one or more network interfaces 1580 for communicating with other devices via a network. Apparatus 1500 may also include one or more input/output devices 1590 that enable user interaction with apparatus 1500 (e.g., display, keyboard, mouse, speakers, buttons, etc.).

Processor 1510 may include both general and special purpose microprocessors and may be the sole processor or one of multiple processors of apparatus 1500. Processor 1510 may comprise one or more central processing units (CPUs), and one or more graphics processing units (GPUs), which, for example, may work separately from and/or multi-task with one or more CPUs to accelerate processing, e.g., for various image processing applications described herein. Processor 1510, persistent storage device 1520, and/or main memory device 1530 may include, be supplemented by, or incorporated in, one or more application-specific integrated circuits (ASICs) and/or one or more field programmable gate arrays (FPGAs).

Persistent storage device 1520 and main memory device 1530 each comprise a tangible non-transitory computer readable storage medium. Persistent storage device 1520, and main memory device 1530, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1590 may include peripherals, such as a printer, scanner, display screen, etc. For example, input/output devices 1590 may include a display device such as a cathode ray tube (CRT), plasma or liquid crystal display (LCD) monitor for displaying information to a user, a keyboard, and a pointing device such as a mouse or a trackball by which the user can provide input to apparatus 1500.

Any or all of the functions of the systems and apparatuses discussed herein may be performed by processor 1510, and/or incorporated in, an apparatus such as PCR apparatus 1540. Further, PCR apparatus 1540 and/or apparatus 1700 may utilize one or more neural networks or other machine learning or deep-learning techniques performed by processor 1710 or other systems or apparatuses discussed herein.

One skilled in the art will recognize that an implementation of an actual computer or computer system may have other structures and may contain other components as well, and that FIG. 15 is a high-level representation of some of the components of such a computer for illustrative purposes.

The foregoing specification is to be understood as being in every respect illustrative and exemplary, but not restrictive, and the scope of the invention disclosed herein is not to be determined from the specification, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

### Terminology

Terminology used herein with reference to embodiments of the present invention disclosed in this document should be accorded its ordinary meaning according to those of ordinary skill in the art unless otherwise indicated expressly or by context.

Allele: each of two or more alternative forms of DNA that are found at the same location on a chromosome

Allele A and allele B: For a SNP or Indel, the two alternatives that can be observed and measured in a given sample are designated as “allele A” and “allele B”

Array: DNA microarray that is used to genotype known genetic variants (SNPs and indels) in the population

Clustering space: The X and Y dimensions defined by Signal Contrast and Signal Size

Exome: ˜1-2% of the human genome that codes for proteins

Genotyping: method for determining the base (A, G, T, or C) or Indel present at a specific location in a person's DNA

Het call: short for heterozygous call

Hom call: short for homozygous call

Heterozygous: two different alleles at a given locus in an individual

Homozygous: two identical alleles at a given locus in an individual

Indel: type of variant where one or more bases are inserted or deleted as compared to the reference genome

Major Homozygous: two identical alleles at a given locus that represent the most common allele at a given locus for the population of interest

Minor Homozygous: two identical alleles at a given locus that represent the less common allele at a given locus for the population of interest

Mean positive predictive value over a set of variants and individuals: the average over the positive predictive values for each variant.

nAB: the number of samples called heterozygous by AxiomGT1 in the clustering space.

Negative predictive value: proportion of the normal alleles that are confirmed by the reference standard (true negative/(true negative+false negative))

Positive predictive value: proportion of variant alleles that are confirmed by the reference standard (true positive/(true positive+false positive))

Overall positive predictive value: Positive predictive value across all variants and samples

Probeset: A specific set (e.g., a group of three or more) of DNA sequences on the microarray that detect the presence of two or more alleles at a given locus (thus interrogating a specific known polymorphic or nonpolymorphic location in the genome).

Sensitivity: proportion of variant alleles detected by the reference standard that are also found by the index test (true positive/(true positive+false negative))

Signal Contrast and Signal Size: AxiomGT1 genotype clustering is carried out in two dimensions. The X dimension is called “contrast” and the Y dimension is called “size”. They are log-linear combinations of the two allele signal intensities. For alleles A and B, contrast is log 2(A/B) and size is (log 2(A)+log 2(B))/2

Single nucleotide polymorphism (SNP): type of single nucleotide variant; a position in the genome where an individual differs from the reference human genome by a single base change (i.e., a substitution of a single letter of DNA). A SNP may be rare or common in the population.

Specificity: proportion of normal alleles detected by the reference standard that are also found to be normal by the index test (true negative/(true negative+false positive))

Variant: Locus in the genome where different alleles have been observed in different people

What is claimed is:

1. A method of genotyping one or more genetic rare variants in a plurality of nucleic acid samples using assay data generated from one or more assays of the plurality of nucleic acid samples on at least one microarray comprising a plurality of probesets, the method comprising:
evaluating, using one or more computer processors, one or more heterozygous genotype calls generated from the assay data to identify any rare heterozygous genotype calls wherein the number of heterozygous genotype calls for the probeset does not exceed a maximum threshold value;
for each identified rare heterozygous genotype call, determining, using one or more computer processors, whether the identified rare heterozygous genotype call is a suspect call and setting some of the suspect calls to a no call value; and
for each of the identified rare heterozygous genotype calls that is not set to a no call value, evaluating the identified rare heterozygous genotype call to determine whether the identified rare heterozygous genotype call comprises a true rare heterozygous genotype call, wherein evaluation comprises using a supervised machine learning classification model executing on one or more computer processors, the supervised machine learning classification model comprising a plurality of predictor values for identifying a true rare heterozygous genotype call, wherein the supervised machine learning classification model is trained using input to one or more computer processors executing the supervised machine learning classification model, the input comprising a plurality of data points, wherein each data point corresponds to one of a discrete set of signals for each of a plurality of training set nucleic acid samples.

2. The method of claim 1, wherein the supervised machine learning classification model comprises a support vector machine prediction model.

3. The method of claim 1, wherein the maximum threshold value for the number of heterozygous genotype calls for the probeset is three.

4. The method of claim 1, wherein the maximum threshold value for the number of heterozygous genotype calls for the probeset is four.

5. The method of any of claims 1-4, wherein for each identified rare heterozygous genotype call, determining whether the identified rare heterozygous genotype call is a suspect call and set to a no call value, further comprises:
testing the identified rare heterozygous genotype call for any unexpected intensities;
retaining the identified rare heterozygous call if there are no unexpected intensities; and
if there are unexpected intensities, performing a further inspection of one or more individual probe intensities to determine whether the rare heterozygous genotype call should be set to a no call value.

6. The method of claim 5, wherein testing the identified rare heterozygous genotype call for any unexpected intensities further comprises:
computing a sequence contrast index (SCI) metric for each allele of each identified rare heterozygous genotype call; and
determining whether the identified rare heterozygous genotype call is a suspect false positive heterozygous genotype call where the SCI metric exceeds a minimum threshold value.

7. The method of claim 6, wherein the SCI metric for each of two alleles A and B for two probe replicates of the probeset comprises:
for the A allele, SCI(A)=(abs(I₁(A)−I₂(A)))/(I₁(A)+I₂(A)); and
for the B allele, SCI(B)=(abs(I₁(B)−I₂(B)))/(I₁(B)+I₂(B)), wherein I₁ and I₂ comprise normalized probe intensities for the two probe replicates.

8. The method of claim 7, wherein a first predictor value of the plurality of predictor values comprises a maximum SCI value that is the maximum of the SCI(A) and SCI(B) values.

9. The method of any of claims 1-8, wherein a second predictor value of the plurality of predictor values comprises a minimum minor standard deviation distance value that is the minimum of the minor standard deviation distance values across all replicated probe sequences i of the probeset, wherein the minor standard deviation distance value for a replicated probe sequence i (MinorSdDist_i) comprises: MinorSdDist_i=[IntensityOfthePutativeHet_i−Mean(HomIntensity_i)]/Std(HomIntensity_i), where IntensityOfthePutativeHet_i comprises a normalized intensity value of the minor homozygous allele of the identified rare heterozygous genotype call at replicated probe sequence i, HomIntensity_i comprises a normalized intensity value of the minor homozygous allele of a homozygous genotype call at replicate probe sequence i, Mean(HomIntensity_i) comprises the mean of HomIntensity_i values over homozygous genotype calls of the probeset, and Std(HomIntensity_i) comprises the standard deviation of HomIntensity_i values over homozygous genotype calls of the probeset.

10. The method of any of claims 1-9, wherein a third predictor value of the plurality of predictor values comprises a maximum major standard deviation distance value that is the maximum of the major standard deviation distance values across all replicated probe sequences i of the probeset, wherein the major standard deviation distance value for a replicated probe sequence i (MajorSdDist_i) comprises: MajorSdDist_i=[IntensityOfthePutativeHet_i−Mean(HomIntensity_i)]/Std(HomIntensity_i), where IntensityOfthePutativeHet_i comprises a normalized intensity value of the major homozygous allele of the identified rare heterozygous genotype call at replicated probe sequence i, HomIntensity_i comprises the normalized intensity value of the major homozygous allele of a homozygous genotype call at replicate probe sequence i, Mean(HomIntensity_i) comprises the mean of the HomIntensity_i values over homozygous genotype calls of the probeset and Std(HomIntensity_i) comprises the standard deviation of HomIntensity_i values over homozygous genotype calls of the probeset.

11. The method of any of the preceding claims, wherein the plurality of predictor values comprises up to three predictor values.

12. The method of any of claims 1-11, wherein the input for the supervised machine learning classification model comprises data points corresponding to at least 9400 training set heterozygous genotype calls.

13. The method of any of claims 2-11, wherein the input for the supervised machine learning classification model comprises a set of data points having a minimum size of 940 training set heterozygous genotype calls.

14. The method of any of claims 1-13, further comprising: running one or more assays of the plurality of nucleic acid samples using at least one microarray comprising a plurality of probesets.

15. The method of any of claims 1-14, further comprising: generating assay data in computer-readable form from results of one or more assays.

16. The method of any of claims 1-15, further comprising: calling, using one or more computer processors, one or more heterozygous genotypes in the plurality of nucleic acid samples based on the assay data.

17. A non-transitory computer readable medium storing instructions that, when executed by one or more computer processors, execute the processing of any of claims 1-16.

18. A computer system comprising one or more processors configured to execute the processing of any of claims 1-16.

19. A computer related product comprising a non-transitory computer readable medium storing one or more instructions that when executed by one or more processors, perform a method of genotyping one or more genetic rare variants in a plurality of nucleic acid samples using data generated from one or more assays of the plurality of nucleic acid samples on at least one microarray comprising a plurality of probesets, the method comprising:
evaluating, using one or more computer processors, the heterozygous genotype calls to identify any rare heterozygous genotype calls that are from a probeset wherein the number of heterozygous genotype calls for the probeset does not exceed a maximum threshold value;
for each identified rare heterozygous genotype call, determining, using one or more computer processors, whether the identified rare heterozygous genotype call is a suspect call and setting some of the suspect calls to a no call value; and
for each of the identified rare heterozygous genotype calls that is not set to a no call value, evaluating the identified rare heterozygous genotype call to determine whether the identified rare heterozygous genotype call comprises a true rare heterozygous genotype call, wherein evaluation comprises using a supervised machine learning classification model executing on one or more computer processors, the supervised machine learning classification model comprising a plurality of predictor values for identifying a true rare heterozygous genotype call, wherein the supervised machine learning classification model is trained using input to one or more computer processors executing the supervised machine learning classification model, the input comprising a plurality of data points, wherein each data point corresponds to one of a discrete set of signals for each of a plurality of training set nucleic acid samples.

20. The computer related product of claim 19, wherein the supervised machine learning classification model comprises a support vector machine prediction model.

21. The computer related product of claim 19, wherein the maximum threshold value for the number of heterozygous genotype calls for the probeset is three.

22. The computer related product of claim 19, wherein the maximum threshold value for the number of heterozygous genotype calls for the probeset is four.

23. The computer related product of any of claims 19-22, wherein for each identified rare heterozygous genotype call, determining whether the identified rare heterozygous genotype call is a suspect call and set to a no call value, further comprises:
testing the identified rare heterozygous genotype call for any unexpected intensities;
retaining the identified rare heterozygous call if there are no unexpected intensities; and
if there are unexpected intensities, performing a further inspection of one or more individual probe intensities to determine whether the rare heterozygous genotype call should be set to a no call value.

24. The computer related product of claim 23, wherein testing the identified rare heterozygous genotype call for any unexpected intensities further comprises:
computing a sequence contrast index (SCI) metric for each allele of each identified rare heterozygous genotype call; and
determining whether the identified rare heterozygous genotype call is a suspect false positive heterozygous genotype call where the SCI metric exceeds a minimum threshold value.

25. The computer related product of claim 24, wherein the SCI metric for each of two alleles A and B for two probe replicates of the probeset comprises:
for the A allele, SCI(A)=(abs(I1(A)−I2(A)))/(I1(A)+I2(A)); and
for the B allele, SCI(B)=(abs(I1(B)−I2(B)))/(I1(B)+I2(B)), wherein I1 and I2 comprise normalized probe intensities for the two probe replicates.

26. The computer related product of claim 25, wherein a first predictor value of the plurality of predictor values comprises a maximum SCI value that is the maximum of the SCI(A) and SCI(B) values.

27. The computer related product of any of claims 19-26, wherein a second predictor value of the plurality of predictor values comprises a minimum minor standard deviation distance value that is the minimum of the minor standard deviation distance values across all replicated probe sequences i of the probeset, wherein the minor standard deviation distance value for a replicated probe sequence i (MinorSdDist_i) comprises: MinorSdDist_i=[IntensityOfthePutativeHet_i−Mean(HomIntensity_i)]/Std(HomIntensity_i), where IntensityOfthePutativeHet_i comprises a normalized intensity value of the minor homozygous allele of the identified rare heterozygous genotype call at replicated probe sequence i, HomIntensity_i comprises a normalized intensity value of the minor homozygous allele of a homozygous genotype call at replicate probe sequence i, Mean(HomIntensity_i) comprises the mean of HomIntensity_i values over homozygous genotype calls of the probeset, and Std(HomIntensity_i) comprises the standard deviation of HomIntensity_i values over homozygous genotype calls of the probeset.

28. The computer related product of any of claims 19-27, wherein a third predictor value of the plurality of predictor values comprises a maximum major standard deviation distance value that is the maximum of the major standard deviation distance values across all replicated probe sequences i of the probeset, wherein the major standard deviation distance value for a replicated probe sequence i (MajorSdDist_i) comprises: MajorSdDist_i=[IntensityOfthePutativeHet_i−Mean(HomIntensity_i)]/Std(HomIntensity_j), where IntensityOfthePutativeHet_i comprises a normalized intensity value of the major homozygous allele of the identified rare heterozygous genotype call at replicated probe sequence i, HomIntensity_i comprises the normalized intensity value of the major homozygous allele of a homozygous genotype call at replicate probe sequence i, Mean(HomIntensity_i) comprises the mean of the HomIntensity_i values over homozygous genotype calls of the probeset and Std(HomIntensity_i) comprises the standard deviation of HomIntensity_i values over homozygous genotype calls of the probeset.

29. The computer related product of any of claims 19-28, wherein the plurality of predictor values comprises up to three predictor values.

30. The computer related product of any of claims 19-29, wherein the input for the supervised machine learning classification model comprises data points corresponding to at least 9400 training set heterozygous genotype calls.

31. The computer related product of any of claims 19-30, wherein the input for the supervised machine learning classification model comprises a set of data points having a minimum size of 940 training set heterozygous genotype calls.

32. The computer related product of any of claims 19-31, further comprising running one or more assays of the plurality of nucleic acid samples using at least one microarray comprising a plurality of probesets.

33. The computer related product of any of claims 19-32, further comprising generating assay data in computer-readable form from results of the one or more assays.

34. The computer related product of any of claims 19-33, further comprising calling, using one or more computer processors, one or more heterozygous genotypes in the plurality of nucleic acid samples based on the assay data.
